(19) United States
(12) Patent Application Publication
(10) Pub. No.: US 2023/0310668 A1
(43) Pub. Date: Oct. 5, 2023

(54) Mask Portable Sterilizing Device

(71) Applicants: Kyli Fykes-Morgan, Indianapolis, IN (US); Forest Fykes, Indianapolis, IN (US)

(72) Inventors: Kyli Fykes-Morgan, Indianapolis, IN (US); Forest Fykes, Indianapolis, IN (US)

(21) Appl. No.: 17/713,191

(22) Filed: Apr. 4, 2022

(51) Int. Cl. A61L 2/07; A61L 2/26

(52) U.S. Cl. CPC A61L 2/07; A61L 2/26

(57) ABSTRACT

The present invention relates to a mask portable sterilizing device capable of sterilizing a mask at all times. The device provides steam at a sufficient temperature to disinfect surgical quality face masks. The device is portable and powered by a mobile power source such as an automobile auxiliary power port or a usb power source such as a battery power storage unit.

### BACKGROUND OF THE INVENTION

The present invention relates to a mask portable sterilizing device capable of sterilizing a mask at all times. The device provides steam at a sufficient temperature to disinfect surgical quality face masks. The device is portable and powered by a mobile power source such as an automobile auxiliary power port or a usb power source such as a battery power storage unit.

### SUMMARY OF THE INVENTION

The present invention relates to a mask portable sterilizer, a mounting unit, a sterilizing unit A power unit which are added to the invention comprising the cover unit. The present invention can be reused by removing contaminants and bacteria attached to disposable and multi-use masks.

The present invention can always sterilize the mask by carrying the sterilizer using a portable battery power source or while in transport in the wearer's automobile. The present invention can remove contaminants such as fine dust attached to the mask by supplying steam to the mask.

The present invention can enhance the sterilization effect of the mask by generating a heated element that can also remove odors. The present invention can increase portability by charging the rechargeable battery by wire or wirelessly. The present invention has steam technology that removes makeup and viruses as well. The mask portable sterilizing device can clean both disposable and cloth masks. The dimensions of the mask portable sterilizing device are small enough to allow for placement within a purse or other portable carriage devices.

### DESCRIPTION OF THE DRAWINGS

FIG. 1 is an inside view of the mask portable sterilizer of the present invention.

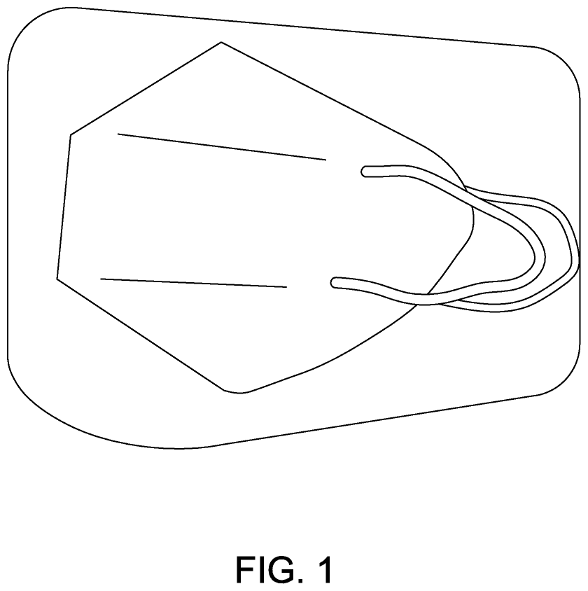

FIG. 3 is an outside and side view of the portable mask sterilizer of FIG. 1;

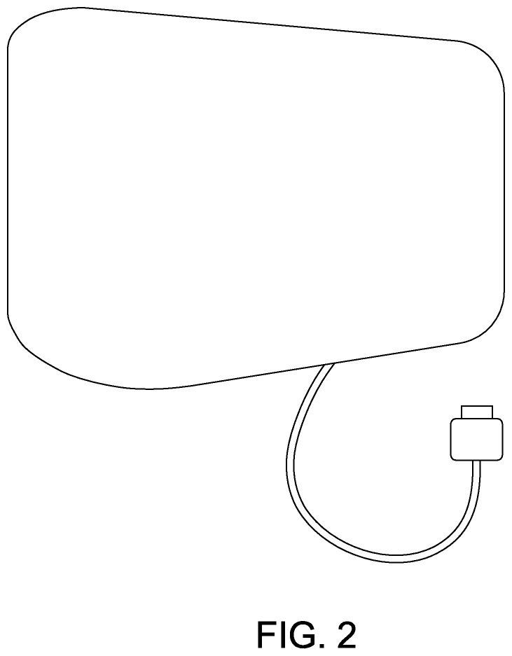

FIG. 3 is a cross-sectional view displaying the water refill tube and steam release section of the mask portable sterilizer of the present invention.

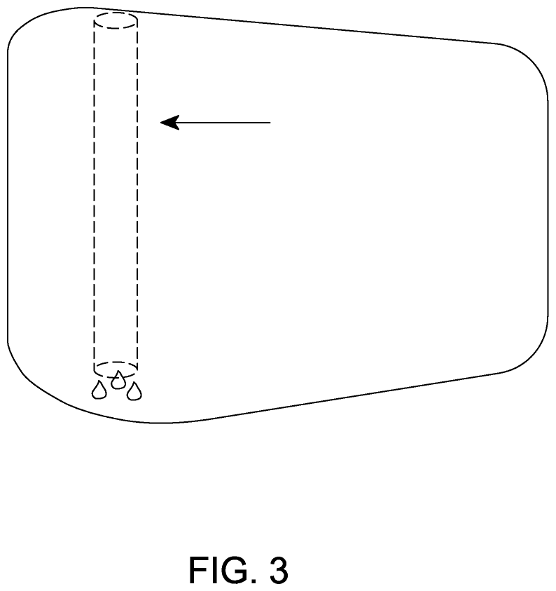

FIG. 4 is a wide view showing the placement of the portable mask sterilizer within an automobile of the present invention.

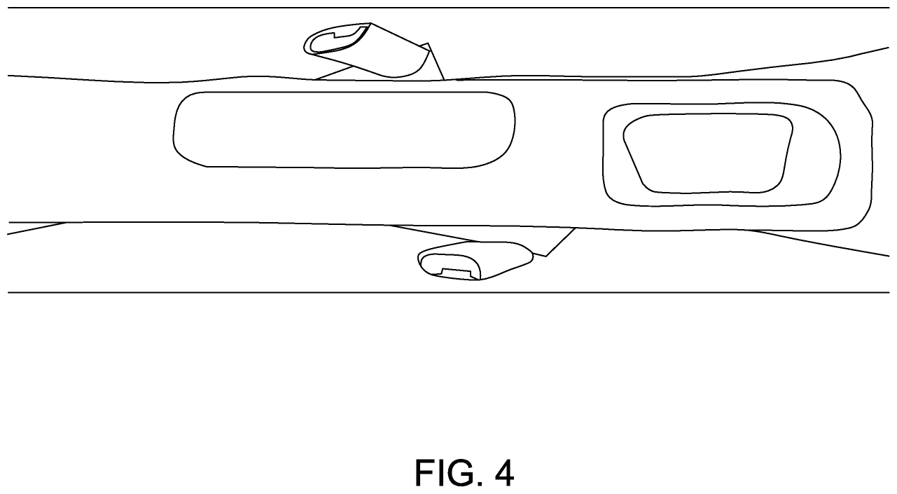

### DETAILED DESCRIPTION

In the present invention, in a mask portable sterilizer, a holding part on which the mask is mounted; is formed at the lower end of the mounting part, and sterilizes the mask a sterilization unit; a cover part rotating from the outside of the sterilization part and surrounding the mounting part. The present invention relates to a portable mask sterilizer. Specifically, it is for sterilizing the mask. In general, the mask is formed of cotton, non-woven fabric, etc., and contaminants or bacteria remaining in the atmosphere are attached to it and flow into the wearer's respiratory tract. This mask has a difficult problem in washing, so it should be discarded after use. Conventional techniques have the disadvantage of inconvenient portability by hanging the mask on a bulky case to sterilize the mask. The mask portable sterilizer of the present invention is to remove fine dust, bacteria, etc. attached to the mask as the wearer uses it, and supplies wind, heat, and ultraviolet rays. This mask portable sterilizer is portable, and when the mask is not in use, the holder for mounting the mask and the sterilization unit is formed. The mounting unit folds the mask in half to mount it in order to increase portability, and the steam supplied from the sterilization unit removes contaminants such as fine dust from the mask, Therefore, the present invention has a feature of carrying a mask portable sterilizer and removing contaminants and bacteria from the mask worn.

I claim:

1. A mask portable sterilizer comprising a mounting unit, a sterilizing unit, a power unit and cover unit;

2. The mask portable sterilizer in claim 1 where the power unit is a portable battery power source;

3. The mask portable sterilizer in claims 1 and 2 where the sterilizing unit comprises a heated element that can also remove odors;

4. The mask portable sterilizer in claim 2 or 3 where the portable battery power source can be charged by wired connection or wirelessly;

5. The mask portable sterilizer in claim 1, 2, 3 or 4 comprising steam technology that removes makeup and viruses.
